(19) United States
(12) Patent Application Publication
(10) Pub. No.: US 2024/0065871 A1
(43) Pub. Date: Feb. 29, 2024

(54) WEIGHT OFFLOADING ORTHOPEDIC DEVICE AND ASSOCIATED METHODS OF USE

(71) Applicant: Raafae M. Hussain, Denton, TX (US)

(72) Inventor: Raafae M. Hussain, Denton, TX (US)

(21) Appl. No.: 18/237,325

(22) Filed: Aug. 23, 2023

Related U.S. Application Data
(60) Provisional application No. 63/401,987, filed on Aug. 29, 2022.

(51) Int. Cl. A61F 5/01

(52) U.S. Cl. CPC A61F 5/0195; A61F 5/0111

(57) ABSTRACT

Disclosed herein are orthopedic devices and methods to aid in reducing weight or pressure on a portion of a subject's foot while maintaining stability of the hindfoot and ankle. Certain embodiments include an upper assembly and a support pillar configured to extend beneath only a portion of a subject's foot. The support pillar can be configured to extend from the heel of a subject's foot to the subject's midfoot, such that at least a portion of the subject's forefoot extends lengthwise therebeyond and does not contact the support pillar. The orthopedic device can further include one or more means for securing the upper assembly around the leg, ankle, dorsal foot, or a combination thereof. Additional aspects include a method of promoting the healing of a podiatric condition using any one or more of the orthopedic devices disclosed herein.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### CROSS-REFERENCE TO RELATED APPLICATIONS

This applications claims the benefit of, and priority to, U.S. Provisional Patent Application No. 63/401,987, filed Aug. 29, 2022, and entitled “Weight Offloading Orthopedic Device,” the entire contents of which is hereby incorporated by reference.

### FIELD OF THE INVENTION

The present disclosure is directed to orthopedic devices and methods. More specifically, the present disclosure related to devices and methods to aid in reducing weight or pressure on a portion of a subject's foot while maintaining stability of the hindfoot and ankle.

### BACKGROUND OF THE INVENTION

Treatment of podiatric injuries, disease, ulceration, and recovery from podiatric procedures such as foot surgery present significant medical challenges, particularly if patient ambulation is expected during the treatment or the recovery process. The human foot contains more than 26 bones, 33 joints, and over a hundred muscles, tendons and ligaments combined. The foot is generally split up into three separate areas; hindfoot, midfoot, and forefoot. The muscles are generally split up into intrinsic and extrinsic, the former having all its origin and insertion at the foot level, and the latter having its origin in the leg and its insertion in the foot. These extrinsic, long muscles originate at the tibia and fibula and can insert anywhere on the foot, including down to the tips of the toes. With traditional casts and boots, the intrinsic muscles are restricted from excessive motion but the extrinsic muscles are in full function, especially whenever walking or standing. Without proper immobilization of these long muscles, the forefoot is open to the push and pull of these extrinsic muscles with normal acts of daily living.

AO (Arbeitsgemeinschaft für Osteosynthesefragen) principles of fracture healing established there are 4 rules for optimal patient outcomes. These rule are restoration of anatomy, stable fracture fixation, preservation of blood supply, and early mobilization of the limb and patient. The first 3 can be performed during surgery, and the last (early mobilization) is left to the patient afterwards. Thus, early mobilization represents elevated risks for negative patient outcomes.

An osseous malunion occurs when a fractured bone heals in an abnormal position and a nonunion is the result of a fractured bone failing to heal after an extended period of time. Fractures or boney surgery in the lower extremity are often complicated by the inherent nature of ambulation. This pressure from simple walking can misalign, or worse, prevent healing of a bone which would normally heal well if it did not receive extrinsic pressures.

With certain forefoot fractures and forefoot surgeries, non-weight bearing is the treatment option of choice, so as not to risk misalignment or nonunion of the bones as they heal. This means any ambulation using standard care casts, surgical boot, or shoes would involve assistive devices, such as crutches, wheelchair, or a knee scooter, while the affected limb is kept non weight bearing.

Aside from post-surgical care, there are many pathologies that would significantly benefit from such a forefoot offloading boot. An example of one such medical challenge is the development of effective treatment options for diabetic foot ulcerations that permit a patient to be independent and ambulatory. Diabetic foot ulcers account for more hospitalizations than any other single complication of diabetes, many of which end with some level of amputation. As such, there is a significant burden placed on the healthcare industry as a result of diabetic foot ulcers and associated infections.

Diabetic neuropathy further adds to this problem by shielding the patient from any sensation of pain. Without the sensation of pain, patients are less likely to voluntarily remain non-weight bearing in the presence of a plantar ulcer. Those who do remain non-weight bearing for an extended period of time often have a low quality of life.

Further, use of standard casts, surgical boots, and shoes can contribute to weight-bearing pressure or friction strain, which exacerbates the risk of postoperative complications such as deep venous thrombosis (DVT), osseous mal unions or non unions, and surgical incision wound dehiscence.

DVTs are blood clots, most commonly forming in the leg. Annually in the United States, more than 200,000 people develop a DVT; and of those, 50,000 cases are also complicated by pulmonary embolism (PE). It is well documented that reduced blood flow (immobility, bed rest, general anesthesia, operations, stroke, long flights, etc.) is one of the major risk factors that can precipitate a DVT.

Reduced ambulation or application of pressure on an affected area can increase the risk of deep venous thrombosis (DVT) or other post-operative complications, such as osseous mal unions or nonunions, and surgical incision wound dehiscence. In addition, proper recovery from certain forefoot fractures and forefoot surgeries requires that the patient avoid bearing weight on the injured area to reduce the risk of misalignment of the bones while healing.

Traditional treatment options such as the use of standard casts or surgical boots/shoes typically distribute the patient's weight across the entire foot, making it nearly impossible for the patient to freely walk during recovery. As a result, the patient cannot move independently, particularly without the use of assistive devices such as crutches, a wheelchair, or a knee scooter, such that the affected limb is kept from bearing weight.

Thus, there is a need for methods and devices that can effectively offload pressure on at least a portion of the foot with the benefit of maintaining ambulatory function.

### SUMMARY OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

In a first aspect, an orthopedic device for offloading weight from a portion of a subject's foot is disclosed herein. In embodiments, the device comprises a single, continuous support pillar, wherein the support pillar is configured to extend across only a portion of a plantar surface of a subject's foot. The device can include a upper assembly extending vertically from the support pillar or a foot covering, wherein the upper assembly is configured to wrap around at least a portion of the subject's leg, at least a dorsal portion of the subject's foot, or a combination thereof.

Embodiments can comprise a foot covering that is configured to enclose at least a portion of the subject's foot therein. Certain embodiments of the device include an outer sole extending distally in the anterior direction from the support pillar. In embodiments, the outer sole can be configured to permit the subject to ambulate without risking injury to the subject's unsupported foot.

In embodiments, the upper assembly further comprises at least one pair of opposing leg flaps and at least one pair of opposing foot flaps. The upper assembly can comprise at least one strut extending vertically from the support pillar and along an outer surface of the upper assembly.

Certain embodiments comprise a means for securing the orthopedic device to the foot of the subject, a means for securing the orthopedic device to the leg of the subject, or a combination thereof. In embodiments the means for securing the orthopedic device to the foot of the subject is configured to join the at least one pair of opposing foot flaps. The device can comprise a means for securing the orthopedic device to the leg of the subject that is configured to join the at least one pair of opposing leg flaps.

In embodiments, the subject's foot comprises a hindfoot, a midfoot, and a forefoot, and the length of the support pillar is sufficient to contact only the hindfoot of the subject and a portion of the midfoot. The length of the support pillar can be sufficient to contact the hindfoot, but not the midfoot and forefoot. In embodiments, the support pillar comprises a bottom portion and a top portion, and the bottom portion of the support pillar comprises a length that is different from a length of the top portion of the support pillar. The length of the bottom portion of the support pillar can be at least about ⅗ the length of the top portion of the support pillar. In another embodiment, the length of the top portion of the support pillar is at least about ⅗ the length of the bottom portion of the support pillar. In embodiments the support pillar further comprises a front side and a back side. The back side of the bottom portion can be substantially orthogonal to the back side of the top portion. In one embodiment, the front side of the top portion extends in the anterior direction beyond the front side of the bottom portion such that the front side comprises an angular slope that extends outwardly from the bottom portion to the top portion. In an alternate embodiment, the front side of the bottom portion extends in the anterior direction beyond the front side of the top portion such that the front side comprises an angular slope that extends outwardly from the top portion to the bottom portion.

In certain embodiments, the support pillar comprises a posterior extension extending lengthwise behind the heel.

The orthopedic device can comprise a first strut and a second strut, wherein the first strut is disposed upon a lateral side of the upper assembly and the second strut is disposed upon a medial side of the upper assembly. The first strut, the second strut, or a combination thereof can comprise a rigid or semi-rigid material.

In various embodiments, the means for securing the orthopedic device to the foot of the subject comprises one or more foot straps, and the means for securing the orthopedic device to the foot of the subject comprises one or more leg straps.

The orthopedic device can comprise a shoe or a boot.

Another aspect of the present disclosure includes a method of off-loading weight to a given portion of a subject's foot comprising any of the various orthopedic device embodiments disclosed herein. In embodiments, the method comprises providing the subject with any of the various orthopedic devices disclosed herein and instructing the subject to wear the device for a given amount of time. The subject can be diagnosed with a podiatric condition, and the given amount of time can comprise the time required for the podiatric condition to substantially heal. In various embodiments, the podiatric condition comprises a break of one or more bones within the foot, a fracture of the foot, soft tissue damage to the foot, a surgical procedure involving the foot, an infection of the foot, an ulceration of the foot, or a combination thereof.

An additional aspect of the present disclosure includes a method of treating a plantar condition in a forefoot of a subject. The method can comprise providing the subject with any of the various orthopedic devices disclosed herein and instructing the subject to wear the device until the plantar condition is substantially healed. In embodiments, the podiatric condition comprises a break of one or more bones within the foot, a fracture of the foot, soft tissue damage to the foot, a surgical procedure involving the foot, an infection of the foot, an ulceration of the foot, or a combination thereof.

Another aspect includes a method of treating a diabetic foot ulceration in a subject. The method comprises providing the subject with any of the various orthopedic devices disclosed herein and instructing the subject to wear the device until the diabetic foot ulceration is substantially healed.

Other objects and advantages of this invention will become readily apparent from the ensuing description.

### BRIEF DESCRIPTION OF THE FIGURES

Certain illustrations, charts, or flow charts are provided to allow for a better understanding of the present invention. It is to be noted, however, that the drawings illustrate only selected embodiments and are therefore not to be considered limiting of scope. Additional and equally effective embodiments and applications of the present invention exist.

FIG. 1 shows a top perspective view of a forefoot offloading boot being worn on a subject's left foot under one embodiment.

FIG. 2 shows a side view of the FIG. 1 embodiment.

FIG. 3 shows a side view of a forefoot offloading boot under another embodiment.

FIG. 4 shows a side view of the FIG. 3 embodiment being worn on a subject's right foot.

FIG. 5 shows a side view of the FIG. 3 embodiment being worn on a subject's right foot. The boot is show in phantom to reveal the subject's foot and leg within the boot, and the subject's hindfoot can be seen on the support pillar of the boot while the forefoot is shown suspended over the outsole.

FIG. 6 shows the side view of the FIG. 5 embodiment being worn on a subject's right foot, but with a portion of the straps and certain lateral embellishments removed. The remaining portions of the boot are show in phantom to reveal the subject's foot and leg within the boot, and the subject's hindfoot can be seen on the support pillar of the boot while the forefoot is shown suspended over the outsole.

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open-term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower)

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below,” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The terms “subject” and “patient,” as used herein, can be used interchangeably and include all members of the animal kingdom including, but not limited to, mammals, animals (e.g., cats, dogs, horses, swine, etc.) and humans.

The term “condition,” whether inflicted via a surgical procedure or naturally from an environmental occurrence or genetic predisposition, as used herein can mean any wound, injury, pathology, infection, infirmity, malady, or any other ailment, particularly those affecting the foot of a subject.

The term “forefoot,” as used herein, can refer to the anatomical portions of a subject's foot that are distal to the tarsal bones. “Forefoot” can comprise the distal-most half of the foot. In embodiments, the forefoot is the distal-most third of the foot. The term “forefoot” can further include the phalanges of the foot or the toes.

As used herein, the term “hindfoot” can refer to the anatomical portions of a subject's foot that is proximal to Chopart's joint.

In embodiments, the term “midfoot” refers to the anatomical portion of a subject's foot that is between the hindfoot and the forefoot.

As used herein, the phrase “anterior direction,” and similar phrases refer to the direction moving from the heal portion of the orthopedic device to the toe portion of the orthopedic device.

The phrase “posterior direction,” and similar phrases as used herein can refer to the direction moving from the toe portion of the orthopedic device to the heal portion of the orthopedic device.

### DESCRIPTION OF SELECTED EMBODIMENTS

Disclosed herein is a device configured to effectively relieve pressure to a portion of a subject's foot. One embodiment comprises a boot that removes forefoot pressure to allow a patient's injury or pathology to heal faster and without interference. Embodiments of the present disclosure provide ongoing and continuous support to a subject while avoiding application of pressure or distribution of weight to at least a portion of the subject's foot. In embodiments, the device is configured to offload weight or pressure from a subject's forefoot while permitting the patient to freely walk without the use of assistive devices such as crutches, a wheelchair, a knee scooter, and other such devices known in the art. The presently disclosed device can be particularly useful during recovery from any given podiatric condition. By way of non-limiting example, the device can be used to assist with or promote recovery from any one or more of the following: a break of the foot, a fracture of the foot, soft tissue damage to the foot, musculoskeletal injury to the foot, a surgical procedure involving the foot, retained hardware in the foot, degenerative changes of the foot, an infection of the foot, and an ulceration of the foot. In various embodiments, the presently disclosed device includes a forefoot off-loading boot that promotes early mobilization in a patient suffering from a podiatric condition (such as a surgical procedure) without risking osseous malunion or non union. Such embodiments can be particularly useful to permit mobilization and pressure on the foot following certain surgical procedures that would otherwise require complete non weightbearing in the recovery period. With a forefoot offloading boot as disclosed herein, any forefoot fracture, forefoot surgeries, or other podiatric condition can be effectively offloaded with the benefit of maintaining near full ambulatory function. This allows for both hands to be open and free functioning, which permits the patient to ambulate as normal, and can inherently expedite healing time versus being bed bound during the healing period.

In various embodiments, the medical device disclosed herein can lead to increased mobility with reduced pressure, which can effectively reduce the risk of postoperative complications such as DVT, osseous malunions or nonunion, and surgical incision wound dehiscence. Indeed, controlled ambulation with one or more of the offloading device embodiments disclosed herein, can mitigate the immobility risk factor and, thus, decrease the risk of conditions such as DVTs

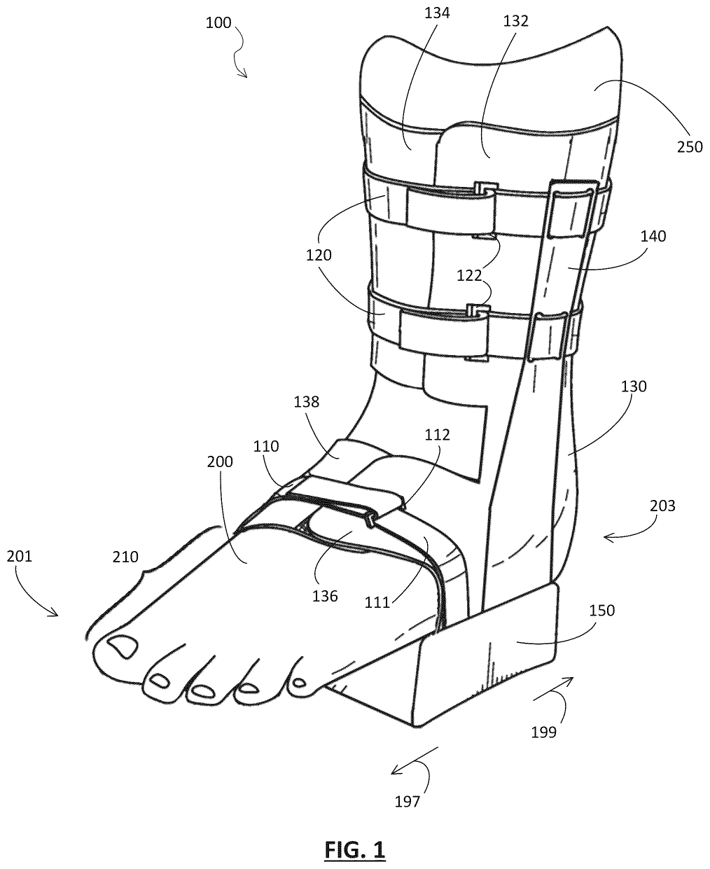

FIG. 1 provides a top perspective view of an orthopedic device 100 under one exemplary embodiment being worn on a subject's left foot 200.

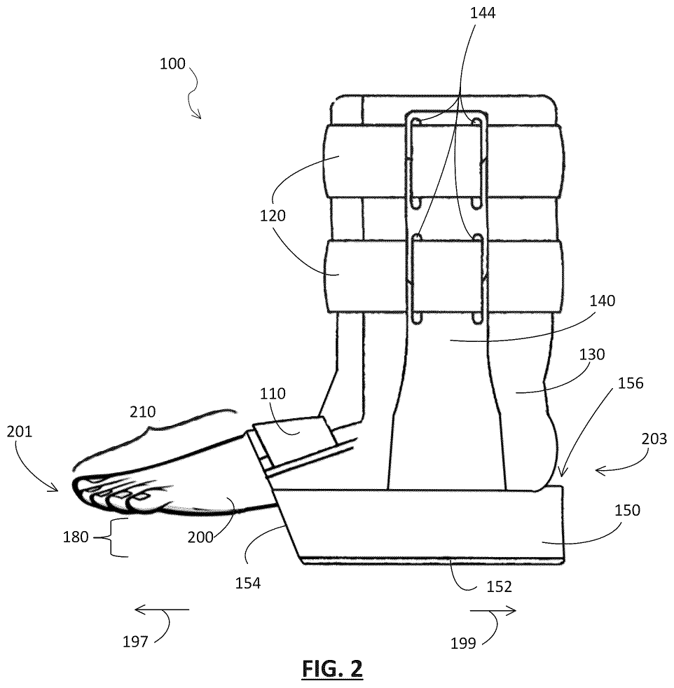

FIG. 2 provides a side view showing the orthopedic device 100 of FIG. 1 from a side view.

As can be seen, a non-load bearing portion 210 of the subject's foot 200 is entirely exposed in the present embodiment, while a weightbearing portion of the foot 200 is concealed within the orthopedic device 100. A distal portion 201 of the subject's foot 200 can be seen near the toes, and a proximal portion 203 of the subject's foot is shown toward the subject's heel.

In the pictured embodiment, the orthopedic device comprises a boot 100 with a hindfoot bed 150, such as a support pillar, and a continuous upper assembly 130 extending upward and vertically from the support pillar 150. The boot 100 can further comprise a pair of rigid or semi-rigid side struts 140 extending upward and vertically from the lateral and medial portions of the support pillar 150.

In embodiments, the upper assembly 130 comprise a continuous piece of material that is configured to at least partially cover a dorsal portion of a subject's foot, the heel of the subject's foot, the sides of the subject's foot, or a combination thereof. In embodiments, the upper assembly 130 does not cover a non-weightbearing portion 210 of the foot 200, such that the non-weightbearing portion 210 is exposed. The upper assembly 130 can comprise at least one pair of opposing overlapping flaps 132, 134, 136, 138 that are configured to assist with closure of the device. Embodiments can comprise up to ten pairs of opposing flaps 132, 134, 136, 138. Embodiments can comprise one, two, three, four, five, six, seven, eight, nine, or ten opposing flaps 132, 134, 136, 138. In embodiments, the upper assembly comprises a pair of opposing leg flaps 132, 134, a pair of opposing foot flaps 136, 138, or a combination thereof. As show in the pictured embodiment, the upper assembly 130 can comprise one or more outer flaps 132, 136 and one or more inner flaps 134, 138, wherein the one or more inner flaps 134, 138 are configured to be disposed underneath the one or more outer flaps 132, 134 when the boot 100 is worn by a subject.

The upper assembly 130 can be configured to extend vertically up at least a portion of a subject's leg 250. In embodiments, the upper assembly 130 extends up to the subject's ankle to form a shoe. The upper assembly can extend above the subject's ankle to form a boot. In certain embodiments, the upper assembly 130 extends vertically at least halfway up the subject's tibia. The upper assembly 130 can be configured to extend up the subject's knee.

In embodiments, the upper assembly 130 may include one or more layers of support material configured to secure or brace the subject's foot 200, leg 250, or a combination thereof. The upper assembly 130 can comprise a soft or pliable material that can be manipulated around at least a portion of the subject's dorsal foot 200, leg 250, or a combination thereof. The upper assembly 130 can also comprise an inner surface that is exposed to the dorsal foot 200 or leg 250 of the subject. In certain embodiments, the inner surface of the upper assembly 130 can comprise a foam or rubber padding configured to provide cushioning to the patient, assist in reducing shearing or sliding of the foot 200 within the boot, or a combination thereof. The upper assembly can comprise a liner 130 that resides between the medial and lateral struts 140 and above the support pillar 150.

In the embodiment of FIGS. 1 & 2, a lateral strut 140 extends upward from the support pillar 150 and is laterally disposed to at least partially support the proximal portion 203 of a subject's foot 200, the subject's ankle, or both. Although only a strut 140 disposed on the lateral side of the boot 100 is visible in the pictured embodiments, the boot 100 can also comprise a medial strut that is disposed on the medial side of the boot 100, wherein the medial side is opposite the lateral side. In embodiments, at least one of the struts 140 comprises an anchor point (seen at 142 of FIG. 2), which is configured to connect the struts 140 to a means for securing the boot 100 to the subject's foot 200, leg 250, or both. The anchor point 140 can comprise a hole, eye, notch, gap, channel, or other opening through which a securing means 110, 120 may be passed or otherwise secured therein or thereupon. In embodiments, the medial strut, lateral strut 140, or a combination thereof are integral with the support pillar 150. In alternate embodiments, the medial strut, lateral strut 140, or both comprise material that is separate and distinct from that of the support pillar 150. The side strut 140 can comprise any material that is known to be suitable as a rigid or semi-rigid support. By way of example, each side strut 140 can comprise a plastic material. The struts 140 can comprise a rigid frame to support the foot; restrict motion in the leg, ankle or foot; or a combination thereof.

The boot 100 can comprise at least one means 110, 111, 120 for reversibly securing the boot 100 to a subject's foot 200, leg 250, or a combination thereof. The securing means 110, 111, 120 can be configured to connect the one or more outer flaps 132, 136 with the one or more inner flaps 134, 138 to reversibly secure the boot 100 to the subject's foot 200, leg 250, or both. In embodiments, the means for reversibly securing the boot 100 to a subject's foot 200 or leg 250 comprises one or more straps with a hook and loop attachment, one or more straps with a buckle attachment, laces, one or more zippers, one or more buttons, or a combination thereof.

In embodiments such as that shown in FIGS. 1 & 2, the means for securing the boot 100 to a subject's foot 200 or leg 250 comprises one or more leg straps 120, one or more foot straps 110, 111, or a combination thereof. Each of the one or more leg straps 120 can comprise a first end that is attached to a lashing hoop 122 and a second end that passes through the anchor point 142 of at least one of the lateral struts 140. The second end of the leg strap 120, can subsequently pass around the back of the leg 250, then optionally, through an anchor point of the medial strut (not picture), and back to the front of the leg 250, wherein the second end of the strap passes through the lashing hoop 122 and can be tightened over the pair of opposing leg flaps 132, 134 and fastened to secure the boot 100 to the leg 250 of the subject.

The foot straps 110, 111 can comprise a first strap 110 and a second foot strap 111. In embodiments, the first foot strap 100 comprises an anchor end and a fastening end, wherein the first foot strap 110 is secured to support pillar 150 at an anchor end. Similarly, the second foot strap 111 comprises an anchoring end that can be attached to the support pillar 150 (as shown in FIG. 1), or a dorsal foot portion of the upper assembly 130 (as show in in FIG. 2). The second foot strap 111 can also comprise a lashing end that is attached to a lashing hoop 112. In such embodiments, the first foot strap 110 can pass over a dorsal portion of the foot, then pass upward through the lashing hoop 112, tightened, and fastened over the pair of opposing foot flaps 136, 138 to reversibly secure the boot 100 to the dorsal midfoot or hindfoot of the subject 200.

The support pillar 150 is partially visible under the subject's foot 200, in FIG. 1, and a full side profile of the support pillar 150 is visible in FIG. 2.

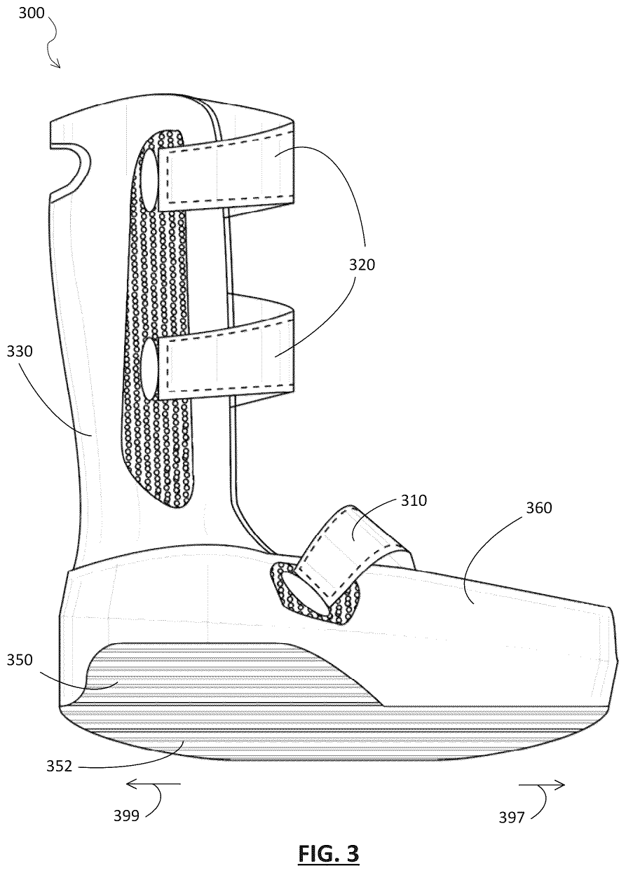

FIG. 3 shows a side view of a forefoot weight offloading boot 300 under another embodiment.

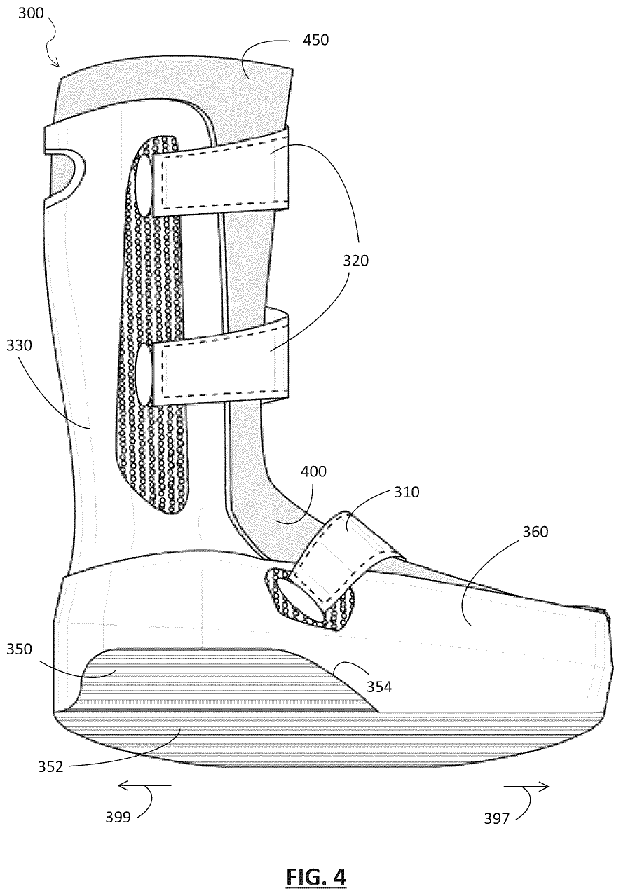

FIG. 4 shows a side view of the FIG. 3 embodiment 300 being worn on a subject's right foot 400.

In the pictured embodiments of FIGS. 3 and 4, the orthopedic device comprises a boot 300 with a hindfoot bed or support pillar 350, a foot covering 360, and a continuous upper assembly 330 extending upward and vertically from a foot covering 360. The boot 300 can further comprise a pair of rigid or semi-rigid side struts that may be integrated within the lateral portions of the upper assembly 330.

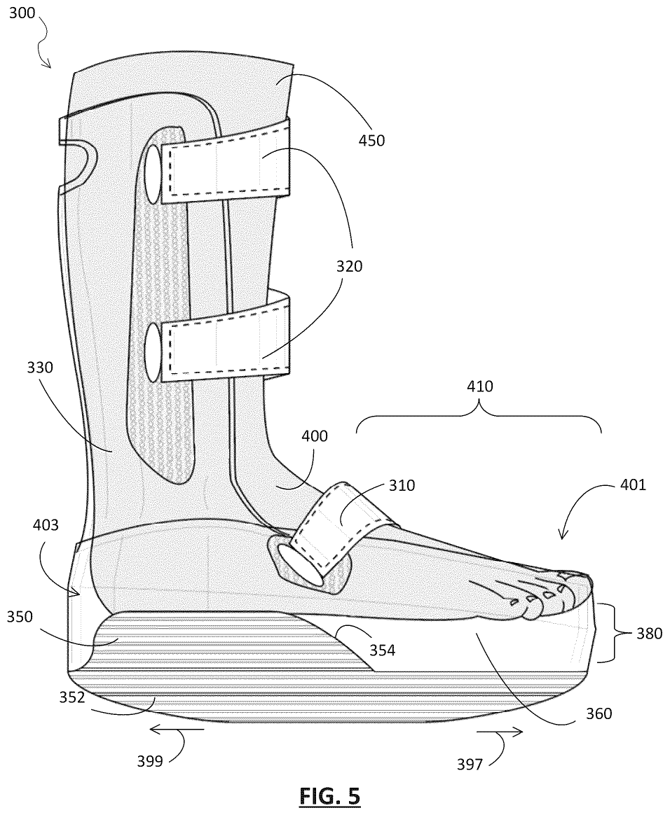

FIG. 5 shows a side view of the FIG. 3 embodiment 300 being worn on a subject's right foot 400. The boot 300 is show in phantom to reveal the subject's foot 400 and leg 450 within the boot 300, and the subject's hindfoot can be seen on the support pillar 350 of the boot 300 while the forefoot 410 is shown suspended at an ambulation height 380 over the outsole 352.

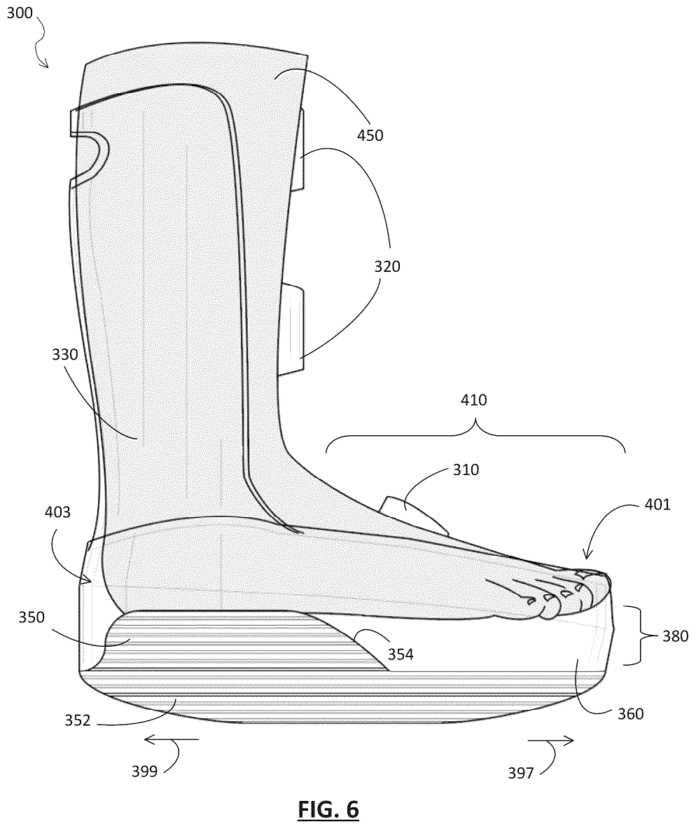

FIG. 6 shows the side view of the FIG. 5 embodiment 300 being worn on a subject's right foot 400, but with a portion of the straps 320, 310 and certain lateral embellishments removed. The remaining portions of the boot 300 are show in phantom to reveal the subject's foot 400 and leg 450 within the boot 300, and the subject's hindfoot can be seen on the support pillar 350 of the boot while the forefoot 410 is shown suspended over the outsole 352.

In FIGS. 5 and 6, a distal portion 401 of the subject's foot 400 is shown near the toes, and a proximal portion 403 of the subject's foot is shown toward the subject's heel. As can be seen, from FIGS. 5 and 6, the proximal-most portions 403 of the subject's foot 400, including the subject's heal, are supported by the support pillar 350 while the distal-most portions 401 of the subject's foot 400 are suspended above the outsole 352. In embodiments, the portions of the subject's foot 400 that are not supported by the support pillar 350 can be referred to herein as the “non-weightbearing portion” 410 of the foot 400, and the portions of the subject's foot 400 that are supported by the support pillar 350 can be referred to as the “weightbearing portion” of the subject's foot 400.

As shown in the embodiments of FIGS. 4-6, the boot 300 can comprise a foot covering 360 that encloses a substantial portion of the subject's foot 400. Such a foot covering 360 can be configured to protect the subject's foot 400 from environmental stimuli while still permitting the boot 300 to prevent the at least a portion of the forefoot from bearing weight. Thus, such embodiments provide many of the protective elements of traditional treatment devices, while avoiding the restriction of movement found in standard medical boots and casts.

In embodiments, the upper assembly 330 comprise a continuous piece of material that is configured to at least partially cover a portion of a subject's foot, the subject's ankle, the subject's leg, or a combination thereof.

The upper assembly 330 can be configured to extend vertically up at least a portion of a subject's leg 450. In embodiments, the upper assembly 330 extends up to the subject's ankle to form a shoe. The upper assembly can extend above the subject's ankle to form a boot. In certain embodiments, the upper assembly 330 extends vertically at least halfway up the subject's tibia. The upper assembly 330 can be configured to extend up the subject's knee.

In embodiments, the upper assembly 330, the foot covering, or both include one or more layers of support material configured to secure or brace the subject's foot 400, leg 450, or a combination thereof. The upper assembly 330, the foot covering 360, or both can comprise a soft or pliable material that can be manipulated around at least a portion of the subject's foot 400, leg 450, or a combination thereof. The upper assembly 330, the foot covering, or both can comprise an inner surface that is exposed to the dorsal foot 400 or leg 450 of the subject. In certain embodiments, the inner surface of the upper assembly 330, the foot covering 360, or both can comprise a foam or rubber padding configured to provide cushioning to the patient, assist in reducing shearing or sliding of the foot 400 within the boot, or a combination thereof. The upper assembly 330 can comprise a liner that forms an internal layer of the boot 300.

In embodiments, a lateral or medial surface of the upper assembly 330, the foot covering 360, or both comprise an anchor point, which is configured to connect the upper assembly 330 to a means for securing the boot 300 to the subject's foot 400, leg 450, or both. The anchor point can comprise a hole, eye, notch, gap, channel, or other opening through which a securing means 310, 320 may be passed or otherwise secured therein or thereupon. The upper assembly 330, foot covering 360, or both can comprise any material that is known to be suitable as a rigid or semi-rigid support. By way of example, the upper assembly 330, foot covering 360, or both can comprise a plastic material. The upper assembly 330, foot covering 360, or both can comprise a rigid frame to support the foot; restrict motion in the leg, ankle or hindfoot; or a combination thereof.

The boot 300 can comprise at least one means 310, 320 for reversibly securing the boot 300 to a subject's foot 400, leg 450, or a combination thereof. As shown in FIGS. 4 and 5, in certain embodiments, the upper assembly 300 can be configured to wrap at least partially around the subject's calf or a posterior portion of the subject's leg. The securing means 310, 320 can be configured to connect around the anterior portion of the subject's leg, a dorsal portion of a subject's foot 400, or a combination thereof such that the boot is reversibly secured to the subject's foot 400, leg 450, or both. In embodiments, the means for reversibly securing the boot 300 to a subject's foot 400 or leg 450 comprises one or more straps with a hook and loop attachment, one or more straps with a buckle attachment, laces, one or more zippers, one or more buttons, one or more elastic straps, or a combination thereof.

In embodiments such as that shown in FIGS. 3-5, the means for securing the boot 300 to a subject's foot 400 or leg 450 comprises one or more leg straps 320, one or more foot straps 310, or a combination thereof. It will be understood by those of skill in the art that the foot straps 310, the at least one leg strap 350, or both can exhibit the same or similar characteristics to those described with references to FIGS. 1 and 2.

As can be seen, in the various embodiments disclosed herein, the length of the support pillar 150, 350 extends across only a portion of the subject's plantar surface of the foot 200, 400, such that none of the subject's weight is applied to at least a portion of the foot 200, 400 when worn by the subject. The support pillar 150, 350 can be configured to extend across the proximal-most half 203, 403 of a subject's foot 200, 400. In embodiments, the support pillar 150, 350 is configured to extend across at least ⅔ of the plantar surface of the subject's foot 200, 400. The support pillar 150, 350 can be configured to extend lengthwise in the anterior direction 197, 397 from the heel to at least a portion of the midfoot of the subject. In certain embodiments, the support pillar 150, 350 only extends across the hindfoot of the subject, such that the midfoot and forefoot do not engage the support pillar 150, 350 and hang freely. In some embodiments, the support pillar 150, 350 is configured to extend across the hindfoot and midfoot such that the forefoot avoids engagement therewith. The support pillar 150, 350 can have a length of up to 10 inches. In embodiments, the length of the support pillar 150, 350 can be up to about 9 inches, up to about 8 inches, up to about 7 inches, up to about 6 inches, up to about 5 inches, up to about 4 inches, up to about 3 inches, or up to about 2 inches. The length of the support pillar 150, 350 can be at least 2 inches, at least 3 inches, at least 4 inches, at least 5 inches, at least 6 inches, at least 7 inches, at least 8 inches, or at least 9 inches. In certain embodiments, the length of the support pillar 150, 350 is between about 1.5 inches and about 10 inches, between about 2 inches and about 8 inches, between about 2.5 inches and about 6 inches, or between about 3 inches and about 5 inches. In embodiments, the length of the support pillar 150, 350 is about 1.50 inches, about 1.75 inches, about 2.00 inches, about 2.25 inches, about 2.50 inches, about 2.75 inches, about 3.00 inches, about 3.25 inches, about 3.50 inches, about 3.75 inches, about 4.00 inches, about 4.25 inches, about 4.50 inches, about 4.75 inches, about 5.00 inches, about 5.25 inches, about 5.50 inches, about 5.75 inches, about 6.00 inches, about 6.25 inches, about 6.50 inches, about 6.75 inches, about 7.00 inches, about 7.25 inches, about 7.50 inches, about 7.75 inches, about 8.00 inches, about 8.25 inches, about 8.50 inches, about 8.75 inches, or about 9.00 inches.

The portion of the subject's foot 200, 400 that is disposed over the support pillar 150, 350 can be considered the weightbearing portion of the foot 200, 400, and the portion of the foot 200, 400 that is not disposed over the support pillar 150, 350 can be considered the non-weight bearing portion 210, 410 of the foot 200, 400. In embodiments, the weight-bearing portion of the foot comprises the hindfoot, at least a portion of the midfoot, or a combination thereof. The non-weight bearing portion 210, 410 can comprise the forefoot, at least a portion of the midfoot, or a combination thereof.

In embodiments, the non-weight bearing portion 210, 410 comprises up to about ⅔ of the subject's foot. The non-weight baring portion 210, 410 can comprise as little as about ⅕ of the subject's foot. The non-weight bearing portion 210, 410 can comprise anywhere between about ¼ to about ⅘ of the subject's foot. In embodiments, the non-weight bearing portion 210, 410 is about ⅓ of the subject's foot. In certain embodiments, the non-weight bearing portion 210, 410 of the subject's foot can be about the same size as the weight bearing portion of the subject's foot. The non-weight bearing portion 210, 410 of the subject's foot can be smaller than the weight bearing portion of the subject's foot. In certain embodiments, the non-weight bearing portion 210, 410 is larger than the weight-bearing portion of the subject's foot. The ratio of the size of the non-weight bearing portion 210, 410 to the size of the weight bearing portion can be about 1:1, about 1.25:1, about 1.5:1, about 1.75:1, about 2:1, about 2.25:1, about 2.5:1, about 2.75:1, or about 3:1. In embodiments, the ratio of the size of the non-weight bearing portion 210, 410 to the size of the weight bearing portion of the subject's foot is about 1:1.25, about 1:1.5, about 1:1.75, about 1:2; about 1:2.25, about 1:2.5, about 1:2.75, about 1:3, about 1:3.25, about 1:3.5, about 1:3.75, or about 1:4.

Advantageously, the support pillar 150, 350 can be configured to support the subject over the weightbearing portion while providing an ambulation height 380 such that subject can maintain full or near full ambulation of the foot 200, 400 without exposing the non-weightbearing portion 210, 410 to pressure, friction, or shear forces that may otherwise interfere with the healing of a podiatric condition disposed on the non-weightbearing portion 210, 410 of the subject's foot 200, 400. In one embodiment, the orthopedic device comprises a forefoot offloading boot 100, 300 that removes or prevents pressure or stress on the forefoot to promote healing of a podiatric condition, such as a break of one or more bones within the foot, a fracture of the foot, soft tissue damage to the foot, a surgical procedure involving the foot, an infection of the foot, and an ulceration of the foot, or a combination thereof. In certain embodiments, the podiatric condition can comprise a condition on the plantar surface of the subject's foot.

In the embodiment of FIGS. 2, 5, and 6, the non-weightbearing portion of the foot is shown elevated at ambulation height 180, 380 above the ground or outsole 152, 352 of the boot 100, 300. In embodiments, the ambulation height 180, 380 is sufficient to permit the subject to curl their toes on the downward direction without the subject's toes coming into contact with the outsole 352 or the ground. The ambulation height 180, 380 can be up to 3 inches. In one embodiment, the ambulation height 180, 380 is up to 2 inches. In embodiments, the ambulation height 180, 380 is up to about 1.5 inches. The ambulation height 180, 380 can be up to about 1 inch. In certain embodiments, the ambulation height 180, 380 is greater than 0.5 inches. The ambulation height 180, 380 can be between about 0.25 inches and about 1.75 inches. In certain embodiments, the ambulation height 180, 380 can be between about 0.5 inches and about 1.5 inches. In embodiments, the ambulation height 180, 380 is about 0.5 inches, about 0.6 inches, about 0.7 inches, about 0.8 inches, about 0.9 inches, about 1.0 inch, about 1.1 inches, about 1.2 inches, about 1.3 inches, about 1.4 inches, about 1.5 inches, about 1.6 inches, about 1.7 inches, about 18 inches, about 1.9 inches, about 2.0 inches, about 2.1 inches, about 2.2 inches, about 2.3 inches, about 2.4 inches, or about 2.5 inches.

In the pictured embodiments of FIGS. 1-6, the anterior direction is denoted by the arrow 197, 397 extending in the direction of the toe, or the front, of the boot 100, 300, and the posterior direction is denoted by the arrow 199, 399 extending in the direction of the heel, or the back) of the boot 100, 300. In various embodiments of the presently disclosed boot 100, 300 the support pillar 150, 350 comprises a posterior extension 156 extending lengthwise in the posterior direction 199 behind the heel 203, 403. The support pillar 150, 350 can comprise a bottom portion that is proximal to the ground or outsole 152, 352 and a top portion, which on a side opposite the bottom portion. The top portion of the support pillar 150, 350 can be configured to be proximal to or to contact the plantar surface of at least a portion of the subject's foot 200, 400 when being worn by a subject.

As shown in the embodiments of FIG. 2, the bottom of the support pillar 150 can comprise a length that is shorter than a top portion of the support pillar 150, 350, and the anterior-most side of the support pillar 150 comprises a flat or angular slope 154 that retracts in the posterior direction 199 from the top of the support pillar 150 to the bottom of the support pillar 150. In embodiments, the bottom of the support pillar 150 is at least about ⅗ the length of the top of the support pillar. The bottom of the support pillar 150 can comprise a length that is up to about 9/10 the length of the top of the support pillar 150. In alternate embodiments, the length of the bottom portion of the support pillar 150 is about the same as the top portion of the support pillar 150. Advantageously, the posterior extension 156, the angular slope 154 or a combination thereof can be configured to encourage the subject to shift their weight to the hindfoot and away from the forefoot.

As shown in the embodiments of FIGS. 4-6, in alternate embodiments, the bottom of the support pillar 350 can comprise a length that is longer than a top portion of the support pillar 350, and the anterior-most side of the support pillar 350 comprises a flat or angular slope 354 that extends in the anterior from the top of the support pillar 350 to the bottom of the support pillar 350. In embodiments, the top of the support pillar 350 is at least about ⅗ the length of the bottom of the support pillar 350. The top of the support pillar 350 can comprise a length that is up to about 9/10 the length of the bottom of the support pillar 350. In alternate embodiments, the length of the top portion of the support pillar 350 is about the same as the top portion of the support pillar 350. Advantageously, the angular slope 354 can be configured to encourage the subject to shift their weight to the hindfoot and away from the forefoot.

The support pillar 150, 350 may comprise a substantially rigid, a semi-rigid material, a soft, foam-like material, or a combination thereof. In embodiments, foam inserts, gel, felt, memory foam, custom molded insoles, heating and cooling pads, medicated pads or any combination thereof can be inserted into the contact surface of the hind foot. In embodiments, the boot can comprise an outsole 152, 352 that is disposed underneath or external to the support pillar 150, 350. In certain embodiments, the support pillar 150, 350 comprises a first material and the outsole 152, 352 comprises a second material, wherein the first material does not comprise the second material, the second material does not comprise the first material, or a combination thereof. In embodiments, the outsole 152, 352 comprises an anti-slip material, a textured surface so as to form a tread, or a combination thereof. By way of example, the anti-slip material can comprise rubber, silicon, or any other, similar material that is known in the art to reduce the risk of slipping during use. It will be understood by those of skill in the art that the boot 100, 300 can be configured for use on the left foot, the right foot, or both feet of a subject in need thereof.

The boot 100, 300 can be configured to fit individuals with a foot size between the 5th percentile of females and the 95th percentile of males, inclusive according to any commonly referenced anthropometric standards. In embodiments, the boot 100, 300 can be configured to fit individuals with a foot size of an average adult male. The boot 100, 300 can be configured to fit the foot of an average adult female. In embodiments, the boot 100, 300 is configured to fit the foot of average-sized 12-year-old child. In certain embodiments, the boot 100, 300 is configured to fit individuals with a foot size of an average-sized toddler.

In embodiments, the orthopedic device 100, 300 is configured to fit on an individual's left foot or an individual's right foot. Certain embodiments are configured to fit both the left and right foot. For instance, the orthopedic device 100, 300 can be configured for use on the right foot and the left foot.

In certain embodiments, the orthopedic device 100, 300 comprises one or more filler pads configured to fill gaps between the device 100, 300 and the subject. The filler pads can be configured to be inserted to remove or reduce any discomfort felt by the subject. In embodiments, the filler pads comprise a means for reversibly securing the pads to the upper assembly 130, 330 the side struts 140, the support pillar 150, 350 or a combination thereof. The filler pads can comprise foam inserts, gel, felt, memory foam, custom molded insoles, heating and cooling pads, medicated pads, or any combination thereof.

Additional embodiments comprise a hinge at or around the level of the subject's ankle. In such embodiments, the side struts 140 can include a pivotal arm. The pivotal arm can be adjusted to the subject's needs (such as comfort or mobility requirements), a practitioner's recommendation, or a combination thereof.

The pivotal arm can be locked at a neutral position to completely limit motion at the foot and ankle in one configuration. In such a configuration, the neural or locked position prevents the foot from moving along the ankle with respect to the leg. In the neutral or locked position, the angle between the foot and the leg can be about 90°. In embodiments, the angle between the foot and the leg at a neutral or locked position is between about 60° and about 120°. The angle between the foot and the leg in the neutral or locked position can be about 70°, about 75°, about 80°, about 85°, about 90°, about 95°, about 100°, about 105°, about 110°, about 115°, or about 120°.

In embodiments, the pivotal arm can be selectively adjusted to different ranges of motion during recovery and progression of the subject's functionality. The pivotal arm can be configured to permit one or more intermediate settings that permit a limited range of motion of the foot relative to the leg. In embodiments, the intermediate setting can permit movement of up to about 60° around the neutral position. The intermediate setting can permit movement of between about 1° and about 50°. In embodiments, the intermediate setting permits movement of about 5°, about 10°, about 15°, about 20°, about 25°, about 30°, about 40°, about 45°, or about 50° around the neutral position. In embodiments, the intermediate position permits movement of about 30° around the neutral position.

In embodiments, the pivotal can be configured to an open setting, which permits full range of motion as needed for ambulation, while still offloading the forefoot.

An alternate aspect disclosed herein is a method of treating a plantar condition of a subject. In embodiments, the method includes providing any of the various embodiments of the orthopedic device 100, 300 disclosed herein to a subject suffering from a plantar condition and instructing the subject to wear the orthopedic device 100, 300 for a given amount of time.

A method of off-loading weight to a given portion of a subject's foot is also disclosed herein. In embodiments of such methods, the subject is provided with any of the presently disclosed orthopedic devices and instructing the subject to wear the device for a given amount of time.

In embodiments comprising a boot 100, 300 the method can include inserting a foot 200, 400 into the upper assembly 130, 330 and sliding the heel of the foot to the back of the boot. The upper assembly 130, 330 can then be closed via the securing means 110, 111, 120, 310, 320 to reversibly secure the boot 100, 300 to a subject's foot 200, 400 leg 250, 450 or a combination thereof. In embodiments, the securing means 110, 111, 310 that are proximal to the toe are secured first, and the securing means 120, 320 surrounding the leg 250, 450 are secured thereafter. Filler pads can be inserted as necessary to relive any discomfort or fill any gaps that may exist between the patient's foot 200, 400 or leg 250, 450 and the boot 100, 300.

A method of treating a diabetic foot ulceration is also disclosed herein. In embodiments, such methods include providing any of the various orthopedic devices disclosed herein to a subject suffering from a diabetic foot ulceration and instructing the subject to wear the orthopedic device on the affected foot until the diabetic foot ulceration is completely healed.

The various methods disclosed herein can further comprises progressively increasing the range of motion via a pivotal arm and an associated hinge. Under such embodiments, the pivotal arm is set to the neutral position for a first period of time during the off-loading period or treatment period. During this first period of time, the foot is kept in the locked position to prevent movement of the foot with respect to the leg. The pivotal arm can be set to an intermediate setting for a second period of time during the off-loading period or treatment period. During the second period of time, the foot is permitted to move over a limited range with respect to the leg. The pivotal arm can be set to an open setting for a third period of time, wherein, during the third period of time, the subject has full or near-full range of motion of the foot with respect to the leg across the ankle joint. In embodiments, treatment includes moving progressively and sequentially through the first period of time, the second period of time, and the third period of time until the subject's podiatric condition is healed.

### EXAMPLES

A shoe or boot primarily used for relieving pressure from the distal half of the foot are disclosed herein. In embodiments, it can be worn by patients who have had recent surgery, fracture, or wound located on the forefoot or midfoot regions of the foot. In various embodiments, the disclosure includes a medical boot for use in supporting a patient's foot and ankle structure comprising of a support pillar which corresponds with the plantar aspect of the heel and an upper assembly with securing straps to partially surround the patient's foot and ankle. The presently disclosed orthopedic device can have a heel raised platform which extends from the posterior aspect of the foot to the midfoot, leaving the forefoot floating off the ground. The ankle can be supported by the boot aspect which will extend halfway up the tibia with securing straps. This is to remove or reduce the risk of injury to the ankle and provide a safer and more stable walking apparatus. A purpose of the boot is to remove any forefoot pressure to allow the appropriate injury or pathology the patient may have to heal faster.

In one embodiment, the present disclosure pertains to a boot specifically designed to aid in the offloading, or reducing weight or pressure, from the distal half of the foot while maintaining stability of the hindfoot and ankle. The boot can comprise a hard plantar support pillar which will only make contact with the patient's heel. The outer covering of the shoe upper, can be designed to be adjustable to secure the patient's lower leg and heel. The forefoot will be free floating, without support, to allow for no pressure to be applied thereon.

One non-limiting purpose of the present invention is to offload the distal half of the foot for the treatment of pressure ulcers, for unstable fractures of the forefoot, and to use post operatively for forefoot procedures. In embodiments, a boot may be employed rather than a shoe to provide stability of a heel lift and additional ankle support, especially in an already compromised patient. In such embodiments, the heel platform can offload the forefoot while the boot provides stability and functionality to the user.

The development of effective treatment options for diabetic foot ulcerations, while allowing functionality to the patient presents as a significant medical challenge. Diabetic foot ulcers account for more hospitalizations than any other single complication of diabetes, many of which end with some level of amputation. There is a significant burden placed on the healthcare industry as a result of diabetic foot ulcers and associated infections.

Diabetic neuropathy further adds to the problem by shielding the patient from any sensation of pain. Without the sensation of pain, patients are less likely to voluntarily remain non weight bearing in the presence of a plantar ulcer. Those who do remain non weight bearing for an extended period of time, often have a low quality of life. A forefoot offloading boot, such as that presently disclosed herein can effectively remove the stress on any plantar forefoot ulcer, giving the ulcer the opportunity to heal. This can also allow for near full ambulatory functionality to the patient.

With certain forefoot fractures and forefoot surgeries, non-weight bearing can be the treatment option of choice, so as not to risk misalignment of the bones as they heal. This means any ambulation using standard care casts, surgical boot or shoes would involve assistive devices, such as crutches, a wheelchair, or a knee scooter, while the affected limb is kept non weight bearing. With a forefoot offloading boot, any forefoot fracture and forefoot surgeries can be effectively offloaded with the benefit of maintaining near full ambulatory function. This allows for both hands to be open and free functioning. This increased mobility with reduced pressure can also effectively reduce the risk of post op complications such as deep venous thrombosis (DVT), osseous mal unions or non-unions, and surgical incision wound dehiscence. All of these problems are exacerbated by weight bearing pressure or friction strain when utilizing standard casts, surgical boots, and shoes. Regardless of how aggressive the treatment plan, the use of effective offloading and stabilization measures will serve to increase the healing potential

FIG. 1 depicts a perspective view of a forefoot offloading boot according to some embodiments of the present disclosure. FIG. 2 illustrates the portion of non-weightbearing as it relates to the forefoot versus hindfoot. It is configured to work as an orthopedic device with aims to offload the distal half of the foot while maintaining stability around the hindfoot and ankle regions. In an embodiment, the boot includes a rigid hindfoot foot bed to which two side strut paddles are coupled. The outer boot includes semi rigid vertical sidewalls, on either side of the ankle, which is attached to the rigid foot bed. The sidewall portion may have foam or rubber padding along its inner surface to provide cushioning to the patient and assist in reducing shearing or sliding of the foot within the boot. The half-length support pillar is configured to facilitate offloading of the patient's forefoot.

The elevated platform may be of varying degrees in length and height to encompass more or less of the foot or depending on the user's shoe size. An embodiment of the invention comprises heel to midfoot extension platform which drops off at the midfoot level, so not to engage the forefoot. The forefoot will be allowed to hang freely without any plantar pressure. The upper can comprise a soft cloth type material with closure straps for retention. The upper assembly may include multiple layers of support material, with the purpose of securing or bracing the patient's limb. The closure straps can engage with the lower leg and dorsal foot, securing the boot to the patient preferred tightness.

### EQUIVALENTS

Those skilled in the art will recognize, or be able to ascertain, using no more than routine experimentation, numerous equivalents to the specific substances and procedures described herein. Such equivalents are considered to be within the scope of this disclosure and are covered by the following claims.

What is claimed:

1. An orthopedic device for offloading weight from a portion of a subject's foot comprising:
a single, continuous support pillar, wherein the support pillar is configured to extend across only a portion of a plantar surface of a subject's foot;
a upper assembly extending vertically from at least a portion of the support pillar, wherein the upper assembly is configured to wrap around at least a portion of the subject's leg, at least a dorsal portion of the subject's foot, or a combination thereof;
a means for securing the orthopedic device to the foot of the subject; and
a means for securing the orthopedic device to the leg of the subject.

2. The orthopedic device of claim 1, the upper assembly further comprising:
at least one pair of opposing leg flaps and at least one pair of opposing foot flaps; and
at least one strut extending vertically from the support pillar and along an outer surface of the upper assembly; wherein:
the means for securing the orthopedic device to the foot of the subject is configured to join the at least one pair of opposing foot flaps, and the means for securing the orthopedic device to the leg of the subject is configured to join the at least one pair of opposing leg flaps.

3. The orthopedic device of claim 1, further comprising a foot covering configured to enclose at least a portion of the subject's foot therein.

4. The orthopedic device of claim 1, further comprising an outsole that is disposed below the support pillar and is configured to extend in the anterior direction beyond the anterior-most portion of the support pillar.

5. The orthopedic device of claim 1, wherein the subject's foot comprises a hindfoot, a midfoot, and a forefoot, and the length of the support pillar is sufficient to contact only the hindfoot of the subject and a portion of the midfoot.

6. The orthopedic device of claim 5, wherein the length of the support pillar is sufficient to contact the hindfoot, but not the midfoot and forefoot.

7. The orthopedic device of claim 1, wherein the support pillar comprises a bottom portion and a top portion, and the bottom portion of the support pillar comprises a length that is different from a length of the top portion of the support pillar.

8. The orthopedic device of claim 7, wherein the length of the bottom portion of the support pillar is at least about ⅗ the length of the top portion of the support pillar.

9. The orthopedic device of claim 7, wherein the length of the top portion of the support pillar is at least about ⅗ the length of the bottom portion of the support pillar.

10. The orthopedic device of claim 7 wherein the support pillar further comprises a front side and a back side;
the back side of the bottom portion being substantially orthogonal to the back side of the top portion; and either:
the front side of the top portion extends in the anterior direction beyond the front side of the bottom portion such that the front side comprises an angular slope that extends outwardly from the bottom portion to the top portion; or
the front side of the bottom portion extends in the anterior direction beyond the front side of the top portion such that the front side comprises an angular slope that extends outwardly from the top portion to the bottom portion.

11. The orthopedic device of claim 1, wherein, the support pillar comprises a posterior extension extending lengthwise behind the heel.

12. The orthopedic device of claim 1, comprising a first strut and a second strut, wherein the first strut is disposed upon a lateral side of the upper assembly and the second strut is disposed upon a medial side of the upper assembly.

13. The orthopedic device of claim 12, wherein the first strut, the second strut, or a combination thereof comprises a rigid or semi-rigid material.

14. The orthopedic device of claim 1, wherein:
the means for securing the orthopedic device to the foot of the subject comprises one or more foot straps; and
the means for securing the orthopedic device to the foot of the subject comprises one or more leg straps.

15. The orthopedic device of claim 1, wherein the device comprises a shoe or a boot.

16. A method of off-loading weight to a given portion of a subject's foot, comprising:
providing the subject with the orthopedic device of claim 1, and
instructing the subject to wear the device for a given amount of time.

17. The method of claim 16, wherein the subject has been diagnosed with a podiatric condition, and the given amount of time comprises the time required for the podiatric condition to substantially heal.

18. The method of claim 17, wherein the podiatric condition comprises a break of one or more bones within the foot, a fracture of the foot, soft tissue damage to the foot, a surgical procedure involving the foot, an infection of the foot, an ulceration of the foot, or a combination thereof.

19. A method of treating a plantar condition in a forefoot of a subject, comprising:
providing the subject with the orthopedic device of claim 1; and
instructing the subject to wear the device until the plantar condition is substantially healed.

20. The method of claim 19, wherein the podiatric condition comprises a break of one or more bones within the foot, a fracture of the foot, soft tissue damage to the foot, a surgical procedure involving the foot, an infection of the foot, an ulceration of the foot, or a combination thereof.
